(19) United States
(12) Patent Application Publication
(10) Pub. No.: US 2004/0224004 A1
(43) Pub. Date: Nov. 11, 2004

(54) Methods and products for bad breath

(76) Inventor: Jan Zielinski, San Diego, CA (US)

Correspondence Address: ROBERT D. FISH, RUTAN & TUCKER LLP, 611 ANTON BLVD 14TH FLOOR, COSTA MESA, CA, 92626-1931, US

(21) Appl. No.: 10/297,376

(22) PCT Filed: Jun. 1, 2001
(86) PCT No.: PCT/US01/17863

(51) Int. Cl. A61K 31/70; A61K 7/16

(52) U.S. Cl. 424/442; 514/23; 424/49

(57) ABSTRACT

Methods and compositions are directed towards a product comprising a food serving that includes at least one of silymarin, an antioxidant, glutathione, a methyl group donor, a sulfur group donor, acetyl CoA, and glucuronic acid in an amount effective to reduce volatile sulfur compounds in breath of an individual at least 10% when the food serving is administered to the individual. Preferred food servings are packaged to form a standard serving size. Thus, particularly contemplated methods comprise a step in which a standard serving size is identified. In a further step, at least one ingredient selected from the group consisting of silymarin, an antioxidant, glutathione, a methyl group donor, a sulfur group donor, acetyl CoA, and glucuronic acid is included in the serving size, and in a still further step, the product is advertised as a treatment of bad breath.

### FIELD OF THE INVENTION

The field of the invention is food products and related methods.

### BACKGROUND OF THE INVENTION

Bad breath is unfortunately relatively common among humans and animals and may be caused by various factors, including certain types of food, inability to properly digest, periodontal diseases.

There are numerous products and procedures available for individuals suffering from bad breath, and generally include products that mask the odor, mouthwash to reduce germs in the mouth, antibiotics for treatment of periodontal infections. Alternatively, surgery maybe indicated to help people properly digest food. However, these products and procedures are often ineffective, painful, and/or expensive. For example, breath mints and flavored chewing gum mask the bad breath without eliminating the cause of bad breath. Similarly, most mouthwashes provide only a temporary relief. On the other hand, antibiotics eliminate with relatively high efficiency at least some of the odor-generating agents, however, are typically relatively expensive and may even lead to adverse side effects (e.g., generation of resistant strains, allergic reactions, etc.).

Besides products and procedures available for use in humans, there are also various products known in the art to reduce bad breath in canines. For example, such products include hard biscuits or raw bones that help remove plaque, or tablets containing natural or synthetic flavors. However, most of these products tend to be ineffective in reducing bad breath for relatively long periods of time. For example, raw bones and biscuits frequently fail to remove plaque effectively and as a result, dogs still have bad breath. Similarly, tablets with masking flavors may provide only temporary relief for a dog's bad breath.

Although there are many methods and products known in the art to reduce bad breath, all or almost all of them suffer from one or more disadvantage. Therefore, there is still a need for improved products and methods to reduce or eliminate bad breath in humans and animals.

### SUMMARY OF THE INVENTION

The present invention is directed to compositions and methods including a food serving with at least one ingredient selected from the group consisting of silymarin, an antioxidant, glutathione, a methyl group donor, a sulfur group donor, acetyl CoA, and glucuronic acid, wherein the ingredient is present in the food serving in an amount effective to reduce volatile sulfur compounds in breath of an individual (e.g., a human or a pet) when the food serving is administered to the individual.

In one aspect, the ingredient is present in the food serving in an amount effective to reduce volatile sulfur compounds in breath of an individual by at least 10%, more preferably by at least 50% and most preferably by at least 75% when the food serving is administered to the individual, and it is further preferred that the food serving provides at least 80% of the daily recommended caloric input of the individual.

In another aspect, the antioxidant is selected from the group consisting of ascorbic acid, alpha-tocopherol, astaxanthin, beta-carotene, and a green tea extract, the methyl group donor is selected from the group consisting of betaine and S-adenosylmethionine, and the sulfur group donor is selected from the group consisting of methionine, cysteine, taurine, ethanethiosulfate, and sodium thiosulfate.

In a further aspect, a method of marketing a product include a step in which a standard serving size is identified. In a further step, at least one ingredient selected from the group consisting of silymarin, an antioxidant, glutathione, a methyl group donor, a sulfur group donor, acetyl CoA, and glucuronic acid is included in the serving size, and in a still further step, the product is advertised as a treatment of bad breath.

Various objects, features, aspects and advantages of the present invention will become more apparent from the following detailed description of preferred embodiments of the invention.

### DETAILED DESCRIPTION

The inventors have surprisingly discovered that bad breath in an individual can be reduced or even prevented by including an active ingredient in a food serving. More particularly, the inventors contemplate a product that comprises a food serving that includes at least one ingredient selected from the group consisting of silymarin, an antioxidant, glutathione, a methyl group donor, a sulfur group donor, acetyl CoA, and glucuronic acid. The ingredient is present in the food serving in an amount effective to reduce volatile sulfur compounds in breath of an individual in an amount of at least 10% when the food serving is administered to the individual, and the food serving is preferably packaged to form a standard serving size.

In a particularly preferred aspect, the product comprises a standard serving size (approximately 400 g (about 13.2 oz)) standard dog chow (e.g., comprising sufficient water for processing, poultry by-products, meat by-products, chicken, sunflower oil, citrus pectin, minerals, guar gum, sodium tripolyphosphate, carrageenan, yam flour, tetra potassium pyrophosphate, caramel coloring, xanthan gum, natural flavors, onion powder, garlic powder, yellow #5, and yellow #6). The dog chow further comprises powdered milk thistle extract standardized to 50 mg of silymarin (e.g., commercially available from TwinLab®) and 500 mg reduced glutathione (e.g., commercially available from Jarrow™ Formulas) as active ingredients, an amount effective to reduce volatile sulfur compounds in breath of an dog by at least 10% when the food serving is administered to the dog.

With respect to the food serving, it is contemplated that numerous food servings other than the exemplary dog chow are also appropriate. In fact, all foodstuffs fit for consumption by an animal (and particularly including a human and a pet) are considered suitable for use in conjunction with the teachings presented herein. For example, where the animal is a dog or a cat, all known dog or cat foodstuffs are contemplated, and especially preferred foodstuffs include dried, canned, and/or freshly prepared animal treats. On the other hand, where the animal is a human, all food types (e.g., snack, meal-on-the-go, full-size lunch or dinner, etc.) fit for human consumption are contemplated.

It is especially preferred, that the amount of contemplated foodstuffs (ie., edible products) is a standard serving size. Depending on the type of foodstuff, the amount of contemplated standard serving sizes may vary. For example, where the foodstuff is an animal treat, the standard serving size may be a dog biscuit. On the other hand, where the foodstuff is cat food, the standard serving size may be a can (e.g., holding approximately 180 g of cat food). However, it is especially preferred that the food serving comprises at least 10%, more preferably 25%, even more preferably 50%, and most preferably 80% of the daily recommended caloric input of the animal.

In further aspects of the inventive subject matter, contemplated active ingredients need not be limited to silymarin and glutathione (reduced form) in the above-specified amount. For example, suitable ingredients may include silymarin in isolated or synthetic form, silymarin extracts of various purities (with respect to silymarin), or oxidized glutathione. Moreover, further contemplated ingredients include one or more antioxidants (e.g., ascorbic acid, alpha-tocopherol, astaxanthin, beta-carotene, and/or green tea extract), one or more methyl group donors (e.g., betaine and S-adenosylmethionine), one or more sulfur group donors (e.g., methionine, cysteine, taurine, ethanethiosulfate, and/or sodium thiosulfate), acetyl CoA, glucuronic acid, or any reasonable combination thereof. With respect to the ingredients, it should be appreciated that all contemplated ingredients are readily available in pure form as well as in food supplement form (e.g., tablet, syrup powder, etc.), and that contemplated ingredients are admixed to the serving size or food serving by any conventional method (e.g., stirring, blending, intermingling, etc.). For example it is contemplated that suitable food servings or serving sizes may include at least two, or at least three contemplated ingredients.

With respect to the amount of contemplated ingredients, it should be appreciated that all amounts are suitable that are effective to reduce total volatile sulfur compounds in breath of an individual in an amount by at least 5%, more preferably 10%, even more preferably 50%, and most preferably at least 75% when the food serving is administered to the individual (e.g., in a single measurement 6 hours after repeated administration of contemplated ingredients). There are numerous known methods of measuring volatile sulfur compounds (VSC) in breath, and all of the known methods are contemplated suitable for use herein. However, an especially preferred method includes gas chromatographic separation and quantification of the VSCs. Especially contemplated VSCs include hydrogen sulfide, mercaptomethane, and mercaptoethane.

While not wishing to be bound by any particular theory or mechanism, the inventors contemplate that the ingredient in contemplated products and food servings stimulates the detoxification process in the liver of the animal to which contemplated products and food servings are administered, and thereby decrease the amount of odor-forming volatile compounds, and especially of contemplated VSCs.

The liver is, among many other functions, thought to play three major roles in detoxification of various compounds. First, the liver filters blood to clear bacteria and other toxins from blood before the blood re-enters the general circulation. Second, the liver produces and secretes bile, which serves among other functions to carry many toxic substances to the intestines where they may be absorbed by fiber and excreted. Third, the liver enzymatically modifies various compounds (and especially toxins) through a two-step process, referred to as phase I and phase II.

Phase I enzymes are thought to neutralize toxins by converting the compounds to an intermediate form that can then be processed by Phase II enzymes. Phase II detoxification involves a system in which enzymes further modify the through conjugation with various cosubstrates. There are several known detoxification pathways in the liver used to conjugate toxins: (1) glutathione conjugation, (2) amino acid conjugation, (3) methylation, (4) sulfation, (5) sulfoxidation, (6) acetylation, and (7) glucoronidation.

Glutathione conjugation involves the conjugation of the tripeptide glutathione, made up of cysteine, glutamic acid, and glycine. Glutathione binds to fat-soluble toxins and converts them to a water-soluble form, allowing the kidneys to excrete the bound toxins more efficiently. In amino acid conjugation, an amino acid binds and neutralizes toxins. Methylation involves conjugating a methyl group to toxins. Most methyl groups are donated by from S-adenosylmethionine (SAM). Sulfation involves conjugating a sulfur-containing compound to toxins. Sulfoxidation involves the process whereby the enzyme, sulfite oxidase, metabolizes sulfites to sulfates, which are then excreted in the urine. Acetylation involves conjugating acetyl CoA to toxins. Glucuronidation involves the combining of glucuronic acid with toxins.

Products to treat improper liver and digestive function have been suggested, including silymarin and betaine. However, it should be appreciated that silymarin has not been suggested to reduce or eliminate bad breath. Similarly, betaine has been suggested to improve bad breath in humans, but there appear to be no suggestions that betaine should be combined with substantial amounts of any other compound for that purpose. Furthermore, it appears that none of the compounds suggested for use in liver toxicity in humans have been suggested to have a desirable effect on bad breath in animals.

Consequently, a method of marketing an edible product may have one step in which a standard serving size of a foodstuff is identified. In a further step, at least one ingredient selected from the group consisting of silymarin, an antioxidant, glutathione, a methyl group donor, a sulfur group donor, acetyl CoA, and glucuronic acid is added to the standard serving size, and in still another step, the product is advertised as a treatment of bad breath. It is further particularly contemplated that the step of advertising may comprise advertising the product as a treatment of bad breath in a human or a pet (e.g., a dog or a cat). Furthermore, the inventors contemplate that contemplated products and food servings my be especially useful for improving liver function, digestions, skin color, and periodontal health. Thus, it should be appreciated that contemplated methods may further comprise advertising the product for improving liver function, digestions, skin color, and periodontal health. Advertising may be performed in all known manners, and particularly includes providing a printed instruction on the product, providing an informative brochure, and providing a broadcast information (e.g., via television, Inernet, or radio).

Thus, specific embodiments and applications of methods and products for bad breath have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A method of marketing an edible product comprising:
identifying a standard serving size;
including in the serving size at least one ingredient selected from the group consisting of silymarin, an antioxidant, glutathione, a methyl group donor, a sulfur group donor, acetyl CoA, and glucuronic acid; and
advertising the product as a treatment of bad breath.

2. The method of claim 1, wherein the step of advertising comprises advertising the product as a treatment of bad breath in a human.

3. The method of claim 1, wherein the step of advertising comprises advertising the product as a treatment of bad breath in a pet.

4. The method of claim 3, wherein the pet is a dog.

5. The method of claim 1, wherein the silymarin is included in a silymarin extract.

6. The method of claim 1, wherein the antioxidant is selected from the group consisting of ascorbic acid, alpha-tocopherol, astaxanthin, beta-carotene, and a green tea extract.

7. The method of claim 1, wherein the methyl group donor is selected from the group consisting of betaine and S-adenosylmethionine.

8. The method of claim 1, wherein the sulfur group donor is selected from the group consisting of methionine, cysteine, taurine, ethanethiosulfate, and sodium thiosulfate.

9. The method of claim 1, wherein at least two ingredients selected from the group consisting of silymarin, an antioxidant, glutathione, a methyl group donor, a sulfur group donor, acetyl CoA, and glucuronic acid are included in the serving size.

10. The method of claim 1, wherein at least three ingredients selected from the group consisting of silymarin, an antioxidant, glutathione, a methyl group donor, a sulfur group donor, acetyl CoA, and glucuronic acid are included in the serving size.

11. The method of claim 1, further comprising advertising the product for improving liver function.

12. The method of claim 1, further comprising advertising the product for improving digestion.

13. The method of claim 1, further comprising advertising the product for improving periodontal health.

14. A product comprising:
a food serving that includes at least one ingredient selected from the group consisting of silymarin, an antioxidant, glutathione, a methyl group donor, a sulfur group donor, acetyl CoA, and glucuronic acid;
wherein the ingredient is present in the food serving in an amount effective to reduce volatile sulfur compounds in breath of an individual in an amount of at least 10% when the food serving is administered to the individual; and
wherein the food serving is packaged to form a standard serving size.

15. The product of claim 14, wherein the ingredient is present in the food serving in an amount effective to reduce volatile sulfur compounds in breath of an individual in an amount of at least 50% when the food serving is administered to the individual.

16. The product of claim 14, wherein the ingredient is present in the food serving in an amount effective to reduce volatile sulfur compounds in breath of an individual in an amount of at least 75% when the food serving is administered to the individual.

17. The product of claim 14, wherein the food serving comprises at least 80% of a daily recommended caloric input of an individual.

18. The product of claim 14, wherein the antioxidant is selected from the group consisting of ascorbic acid, alpha-tocopherol, astaxanthin, beta-carotene, and a green tea extract, wherein the methyl group donor is selected from the group consisting of betaine and S-adenosylmethionine, and wherein the sulfur group donor is selected from the group consisting of methionine, cysteine, taurine, ethanethiosulfate, and sodium thiosulfate.

19. The product of claim 14, wherein the food serving includes at least three ingredients selected from the group consisting of silymarin, an antioxidant, glutathione, a methyl group donor, a sulfur group donor, acetyl CoA, and glucuronic acid are included in the serving size.

20. The product of claim 14, wherein the product is an animal treat.
